(19) United States
(12) Patent Application Publication
(10) Pub. No.: US 2009/0047297 A1
(43) Pub. Date: Feb. 19, 2009

(54) MICROFLUID SYSTEM FOR THE ISOLATION OF BILOGICAL PARTICLES USING IMMUNOMAGNETIC SEPARATION

(76) Inventors: Jungtae Kim, Dudweiler (DE); Ute Steinfeld, St. Ingebert (DE); Jorg Schuhmacher, St. Ingebert (DE)

Correspondence Address: BARNES & THORNBURG LLP, 11 SOUTH MERIDIAN, INDIANAPOLIS, IN, 46204, US

(21) Appl. No.: 11/660,778

(22) PCT Filed: Aug. 22, 2005
(86) PCT No.: PCT/EP2005/009065

§ 371 Date: Oct. 31, 2008

(30) Foreign Application Priority Data
Aug. 23, 2004 (DE) 10 2004 040 785.1

(51) Int. Cl. A61K 39/00; B03C 1/021

(52) U.S. Cl. 424/184.1; 210/222; 210/137; 210/695

(57) ABSTRACT

The present invention relates to a device and to a method for the isolation of biological particles. This device has a throughflow channel 5 and also a first and second magnetic field and also two inlet channels 1, 2 and two outlet channels 3, 4. The first magnetic field is disposed downstream of the inflow region of the inlet channels laterally of the throughflow channel 5, the second magnetic field 7 downstream of the first magnetic field 6 and on the oppositely situated side of the throughflow channel 5. The two magnetic fields can be produced also by a single magnet in a suitable arrangement.

The present invention relates to a device and a method for the isolation of biological particles. There should be understood by biological particles (termed subsequently also alternatively as biological materials), particles or materials on a particulate or molecular basis. There are, included here cells, such as for example viruses or bacteria, in particular however also isolated human and animal cells, such as leucocytes or tumour cells, and also low molecular and high molecular chemical compounds, such as proteins and molecules, in particular immunologically active compounds, such as antigens, antibodies and nucleic acids or also antigen-specific tetramers, such as for example MHC tetramers or also streptamers. The present invention relates in particular to immunomagnetic separation techniques (IMS) for human or animal cells, automatic sample preparation techniques and also (electro)magnetic or magnetic separation techniques (EMS) and microfluid techniques. The immunomagnetic separation techniques are implemented using immunomagnetic particles. There are understood by immunomagnetic particles, magnetisable or magnetic, for example ferromagnetic or superparamagnetic particles or also soft magnetic materials, such as for example ferrites which are characterised (for example by coupling with an antibody or an antigen-specific tetramer) such that they are capable of specific binding to a specific biological material or to a specific biological particle. The immunomagnetic particles which are capable of binding preferably have essentially a spherical form (and therefore are alternatively termed subsequently also as immunomagnetic balls or antibody-coupled magnetic balls) and preferably have particle sizes of less than 100 μm.

Because of the different immune characteristics of biological particles, specific particles (for example antigens or antigen-specific tetramers or streptamers) can be characterised by specific antibodies or bound to specific antibodies (immune reaction or antigen-antibody reaction).

Structures which comprise four MHC molecules and antigens are termed as tetramers in immunology. T-cells bind to these structures one thousand times better than to the individual complexes. The tetramers thereby bind to the corresponding T-cell receptors. This corresponds to the T-cell-mediated, secondary immune response by recognition of cell-bound antigens, in the form of peptides, which are bound by MHC complexes to antigen-presenting cells.

In the meantime, recombinant, soluble MHC molecules can be produced and be bound to a known antigen and can be tetramised by streptavidin. The thus produced peptide-specific tetramer MHC molecules can be marked with fluorescence colourants and be used for measurements in a flow cytometer. By means of the tetramer technique, frequencies of antigen-specific T-cells can be determined in order hence to be able to obtain evidence about the antigens involved in the symptoms of an illness. By means of MHC molecules it is possible to sort and analyse for example T-cells which recognise tumour antigens. Peptide-MHC tetramers have therefore great therapeutic potential in the tracing of antigen-specific T-cells in human autoimmune diseases, for example arthritis.

Binding of the tetramers to particles would ensure better binding of antigen-specific T-cells to the particles which can then be separated in turn, because of the particles, from remaining, non-bound cells.

Reversible MHC-peptide multimers, so-called streptamers, are a new technology for the preparation and isolation of cytotoxic T-lymphocytes. In contrast to the tetramers used to date, they can be separated again from the T-cells and hence do not affect the function of the cells.

If these particles are bound to magnetic balls, then, because of an immune-specific reaction, biological particles coupled to these particles then have, as bound biological particles, likewise magnetic, preferably superparamagnetic or ferromagnetic properties. Hence by using magnets, for example electromagnets or permanent magnets, biological particles which are thus bound in such to antibodies coupled with magnetic particles can be separated and isolated.

It is the object of the present invention to make available a separation device which operates in the throughflow method or a corresponding separation method, with which automatic and continuous isolation of biological particles is possible in a simple manner.

The device according to the invention, in order to achieve this object, uses a simple microfluid channel having two inlets or inlet channels and two outlets or two outlet channels and also one or more magnets, for example electromagnets or permanent magnets. There is understood in the following by a channel (this applies both to the throughflow channel and to the inlet channels opening into said throughflow channel and the discharge channels guided away from said throughflow channel) a volume including the wall surrounding this volume which is subject to a flow by a fluid.

A liquid which contains different biological and/or also non-biological materials (including the biological particles to be determined via the specific immune reaction) is introduced through the first inlet channel into the microfluid throughflow channel. A liquid which contains the immunomagnetic particles which are configured for specific binding to the biological material to be determined is introduced through the other inlet channel. The specific binding can be achieved in that the biological material to be separated by means of the immune reaction is an antigen and in that the immunomagnetic particles are ferromagnetic or superparamagnetic balls which are bound to the corresponding antibody or to antigen-specific tetramers or streptamers (antigen-antibody/tetramer/streptamer reaction).

The rheological properties of the two liquids and also the geometric ratios (in particular the cross-sectional areas of the two inlet channels and also the cross-sectional area of the throughflow channel) are now configured such that the liquid flows supplied through the two inlet channels do not intermix in the throughflow channel (apart from diffusion processes). This can also be achieved in that a separating wall is provided between the region of the inlet channels and the region of the outlet channels in the throughflow channel in such a manner that the respectively supplied or discharged liquid flows are in contact merely in the region of the inlet channels and in the region of the outlet channels. As a result, undesired diffusion effects between the flows are minimised and an even purer separation of the biological particles to be separated is possible.

With the help of the first magnet (or the magnetic field or field gradient thereof), the immunomagnetic particles now obtain in the region of the inlet channels a speed component perpendicular to the flow direction as a result of their ferromagnetic or superparamagnetic character. The immunomagnetic particles can consequently overcome the boundary of both laminar flows or are drawn from one liquid flow into the other liquid flow. In the latter there are then the specific biological particles to be separated, to which the immunomagnetic particles bind. By means of the suitably disposed first magnet or a further second magnet disposed downstream, in the region of the outlet channels, the immunomagnetic particles which are bound at least partially to the biological particles to be separated are then drawn back again, by applying an oppositely directed magnetic field or field gradient, into the original liquid flow. The liquid flow which contains the immunomagnetic particles which are bound to the biological material to be separated is then discharged via one of the outlet channels, whilst the other liquid flow (which contains the remaining biological and/or non-biological materials and non-bound particles of the biological material to be separated) is discharged with the help of the other outlet channel.

It is crucial that laminar flow conditions are present in the microfluid throughflow channel as a result of the conditions prevailing there (rheological properties of the liquids and also in particular the cross-sectional area of the channel). For this reason, the two liquid flows do not intermix or only insubstantially. Hence only the immunomagnetic particles essentially overcome the boundary between the two liquid flows with the help of the first magnetic field and the bound and also the non-bound residual immunomagnetic particles overcome the boundaries of the two liquid flows again in the opposite direction with the help of the magnetic field of the second electromagnet. The immunomagnetic particles are hence introduced separately to the liquid containing the biological particles to be separated, then change for a specific period of time from their liquid flow into the adjacent liquid flow of the biological materials, bind there to the biological particles to be separated and subsequently, with the help of the second magnetic field, are drawn with the biological particles bound to them back again into their original flow. The liquid which contains the non-bound biological particles and also other biological materials is then discharged via the one outlet or discharge channel, whilst the bound and hence isolated biological particles can be discharged from the other outlet.

In an advantageous embodiment variant, the device according to the invention can be provided with a reaction chamber. This is disposed on the throughflow channel on the side of the liquid flow which contains the biological materials or of the first magnet and serves to extend the time which this liquid flow requires to flow through the throughflow channel. The reaction chamber is disposed in the flow direction between the two magnets so that an increased length of stay of the immunomagnetic particles drawn into the flow results and hence a higher probability of the immunomagnetic particles binding to the specific biological material.

The above-described immunomagnetic separation device has a series of advantages:
The device enables simple isolation, without additional mixing, incubation and washing steps which otherwise would require to be implemented by hand and consequently are time-consuming and also require additional liquids. With the device automatic and continuous particle isolation or separation is possible, only small quantities or no quantities at all of buffer, transport and/or dilution liquid being necessary. Sample diluting solutions and additional buffer solutions are hence unnecessary in the present device.
The bound biological particles can hence be isolated and separated without additional washing-out processes from the original mixed liquid which contains various biological materials. The separated biological particles are obtained via a separate discharge channel.
The antibody-coupled magnetic particles or immunomagnetic particles can be supplied directly to their associated inlet channel without in addition a pre-mixing step or an incubation step being required.
The device can be provided with an automatic control device for controlling the magnetic field strengths or magnetic field gradients. Furthermore the device can also be provided with a regulating device which regulates the control of the throughflow rate in the throughflow channel or the liquid quantities flowing through per unit of time. Regulation of the throughflow rate or the quantity of liquid flowing through per unit of time can also be effected by suitable regulating devices in the region of the inlet channels and/or outlet channels. Hence the marking or binding of biological particles and their isolation is possible in a simple and controlled manner.

The device according to the invention can be used as a medical diagnosis system within or outwith the human or animal body. In an equally simple manner, the device according to the invention can also be used for therapeutic purposes, e.g. for isolation of specific types of cells from the blood or tissue of patients and the like. The device can hence be in particular implantable and enable continuous separation or measurement processes. In particular for an implantable device, the latter and also its electronic control unit can be manufactured in an integrated manner and hence have a dimension which is suitable for implantation and be manufactured in an economical manner. If the device according to the invention is used outwith the human or animal body, then it can be configured as a laboratory appliance. The laboratory appliance can be used then for cell separation for example of blood samples, mixed cell populations (e.g. from patient tissue) or of cells with specific characteristics (e.g. specific surface markers or physiological states).

The device according to the invention can be constructed or used as illustrated in one of the two following examples.

FIG. 1 shows a first immunomagnetic separation device according to the invention;

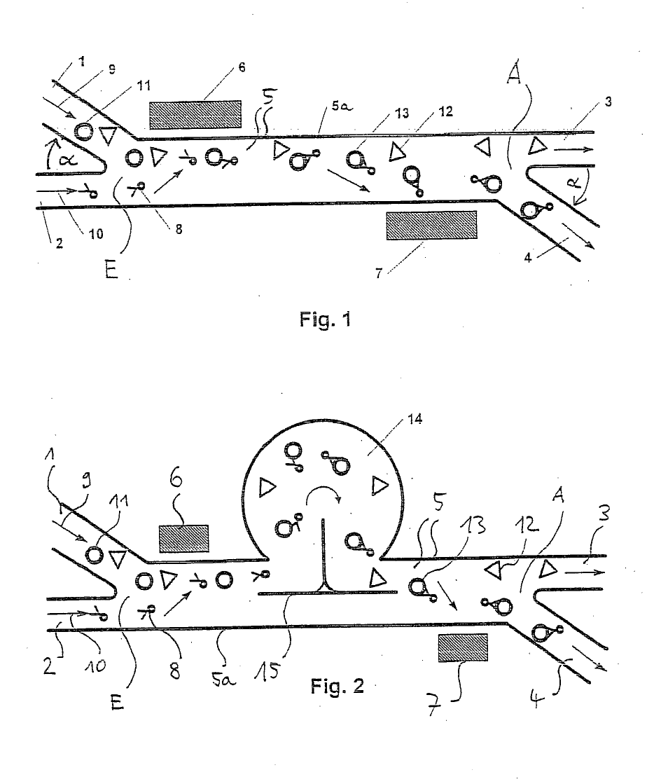

FIG. 2 a second immunomagnetic separation device according to the invention with a reaction chamber;

FIG. 3 a third immunomagnetic separation device according to the invention; and

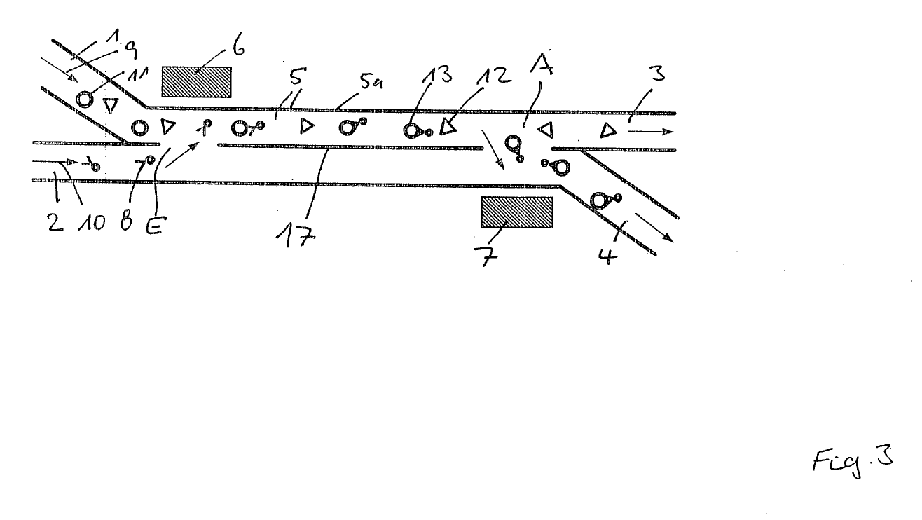

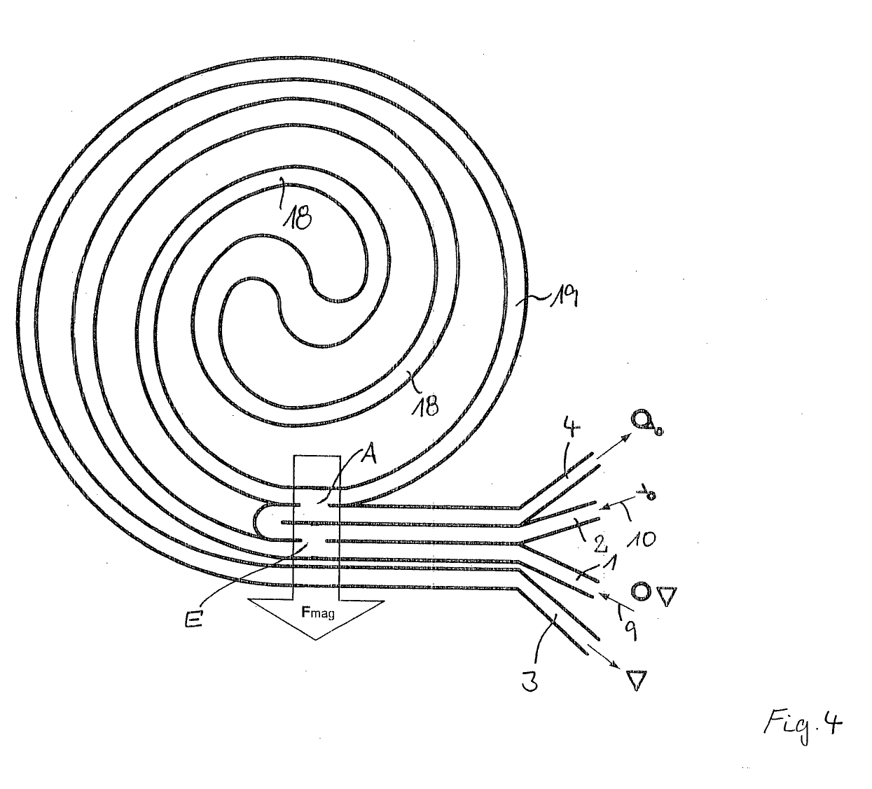

FIG. 4 a further fourth immunomagnetic separation device according to the invention.

In the subsequently described Figures which correspond to the examples, identical reference numbers are used for similar or identical components of the device.

FIG. 1 shows an immunomagnetic separation device. FIG. 1 shows a section through an immunomagnetic separation device according to the invention in a central plane which extends through the centre of gravity of the device. The device has a microfluid throughflow channel 5 with an inflow region E and a discharge region A which is disposed downstream thereof. In the inflow region E, a first inlet channel 1 and a second inlet channel 2 open into the throughflow channel 5. The second inlet channel hereby opens in the direction of the flow direction through the throughflow channel 5. The first inlet channel 1 opens at an angle of α=30° relative to the throughflow direction through the throughflow channel 5. In the discharge region A, two discharge channels 3 and 4 lead out of the throughflow channel 5. The discharge channel 3 hereby leads away in the direction of the flow direction through the throughflow channel 5, the discharge channel 4 leads away at an angle of α=30° relative to this direction. The diameter of the inlet channels 1, 2 and of the discharge channels 3, 4 perpendicular to the respective throughflow direction is approximately half the diameter of the throughflow channel 5 perpendicular to the throughflow direction thereof.

Downstream of the inflow region E, a first electromagnet 6 is disposed outwith the throughflow channel 5 and laterally next to the throughflow channel 5. Downstream of this first electromagnet 6 and directly upstream of the discharge region A, a second electromagnet 7 is likewise disposed outwith the throughflow channel 5 and laterally next to the throughflow channel 5. The two electromagnets 6 and 7 are disposed on different sides, in the present case on oppositely situated side of the throughflow channel 5.

The two electromagnets 6 and 7, alternatively hereto, can however also be integrated at least partially into the wall 5a of the throughflow channel 5. In this case, the two electromagnets 6 and 7 are then integrated on essentially oppositely situated sides in the wall 5a of the throughflow channel 5. It is however also possible to dispose the two electromagnets 6 and 7 entirely within the throughflow channel 5 or within the wall 5a of the throughflow channel 5 in the volume of the throughflow channel 5 which is enclosed by the wall 5a. The two electromagnets 6 and 7 are then likewise disposed within the throughflow channel 5 essentially on oppositely situated sides of the throughflow channel (this takes place preferably in the wall region of the throughflow channel or even such that the electromagnets 6 and 7 are positioned on the inner wall of the channel or are mounted there). It is however also possible to use respectively a different variant from that described for the electromagnet 6 and the electromagnet 7: thus the electromagnet 6 can be disposed entirely outwith the wall 5a of the channel, whilst the electromagnet 7 is integrated on the oppositely situated side of the throughflow channel 5 in the wall thereof or is positioned within the channel on the oppositely situated side on the inner surface of the wall 5a.

The inlet channels 1, 2, the discharge channels 3, 4, the throughflow channel 5 and also the two electromagnets 6 and 7 (or the corresponding central axes or centres of gravity) are disposed in one plane in the present case.

It is now crucial that the conditions in the flow channels, because of sufficiently small diameters of the inlet channels, outlet channels and of the throughflow channel and also because of sufficiently low flow rates, are formed such that two liquid flows or liquid layers which slide separately one above the other can be formed without turbulence (laminar flow). If hence a mixed liquid 9 which contains various biological particles 11, 12 is introduced through the first inlet channel 1 and, through the second inlet channel 2, a liquid 10 which contains immunomagnetic particles 8, then the two introduced liquid flows do not intermix (apart from diffusion processes) but slide in the direction of the discharge region A as separate liquid layers which are parallel to each other. The first liquid flow of the mixed liquid 9 is then discharged via the first discharge channel 3 without intermixing with the second liquid flow 10 of immunomagnetic particles 8, the second liquid flow 10 correspondingly via the second discharge channel 4.

It is therefore crucial that, in the microfluid throughflow channel 5, the throughflowing liquids have such a small Reynolds' number that the flow conditions in the throughflow channel 5 can be regarded as laminar. Hence effects of inertia, which cause turbulences and secondary flows or vortices, are negligible and intermixing is possible solely as a result of diffusion processes. In order to ensure this, the microthroughflow channel. 5 in the illustrated case has a width of 0.1 to 0.3 mm and a height of 0.1 to 0.2 mm (rectangular throughflow channel, width and height perpendicular to the longitudinal direction or to the throughflow direction). The total throughflow rate (regulated via a regulating device, not shown) is between 1 and 200 μl/min for the microthroughflow channel 5. These microfluid flow characteristics fulfil the necessary prerequisites for laminar flow conditions in the microthroughflow channel 5. For this reason, the mixed liquid 9 introduced via the first inlet channel 1 and the liquid 10 which is introduced via the second inlet channel 2 and contains the immunomagnetic particles 8 do not intermix in the throughflow channel 5 but instead form two separate flow layers. Hence also the different particles (biological particles 11, 12 and immunomagnetic particles 8) of each liquid flow are not intermixed when the electromagnets 6, 7 are switched off, but flow continuously in their respective liquid flow up to their respective discharge channel 3 or 4.

In addition to the biological particles 11 to be separated, the mixed liquid 9 in the present case contains further biological (or even different) particles 12, from which the particles 11 to be separated are intended to be separated. Such further particles 12 need not however be present so that the present invention can be used also for altering the concentration of the particles 11 to be separated in the liquid flow 9. If now the first electromagnet 6 is activated, then the immunomagnetic particles 8 are subjected to an electromagnetic field or field gradient which exerts a force perpendicular to the throughflow direction through the throughflow channel 5 and in the direction towards the first electromagnet 6. As a result, the immunomagnetic particles 8 are drawn out of their second liquid flow 10 over the liquid flow boundary into the first liquid flow 9 of the mixed liquid. The immunomagnetic particles 8 hence intermix with the particles 11, 12 situated in the mixed liquid flow 9 and hence can bind to the particles 11 to be separated due to the specific antigen-antibody reaction (hence combined or bound particles 13 are produced, which respectively have at least one immunomagnetic particle 8 and one biological particle 11). The field strength or the gradient strength of the electromagnet 6 can be controlled or adjusted such that the forces which are produced are just sufficient to draw the immunomagnetic particles 8 from the second liquid flow 10 into the first liquid flow 9. The magnetic field of the electromagnet 6 (this applies likewise for the electromagnet 7) can hereby be modulated in a pulsated or sinusoidal form. The immunomagnetic particles then flow freely with an equilibrium condition between the flow rate in the throughflow direction and the speed induced by the magnetic field perpendicularly thereto.

After the immunomagnetic particles 8 have been drawn into the first liquid flow of the mixed liquid 9, as described already, due to an immune-specific reaction, they combine with the biological particles 11 to be separated to form the bound particles 13. The narrowness or the small cross-sectional area of the microthroughflow channel 5 (sufficiently small diameter) and sufficiently low throughflow rates through the throughflow channel 5 increase the probability that the individual immunomagnetic particles 8 bind to the associated biological particles 11 (increase in the time which is available for the immune reaction).

On the downstream side relative to the first electromagnet 6, the second electromagnet 7 is now disposed directly in front of the discharge region A on the side of the throughflow channel 5 situated opposite this magnet. With the help of this second electromagnet 7, the bound particles 13 and also immunomagnetic particles 8 which have not bound to the biological particles 11 on the flow path between the electromagnet 6 and the electromagnet 7 are drawn back again over the liquid flow boundary into the second liquid flow 10. This takes place via an electromagnetic field or a field gradient of the electromagnet 7 which is directed opposite to the field or gradient of the first magnet 6. The immunomagnetically bound or characterised biological particles 13 and also the non-bound immunomagnetic particles 8 or the second liquid flow 10 is then discharged via the second discharge channel 4. The first liquid flow 9 or the remaining non-bound biological particles 11 and also the other biological materials 12 are discharged via the first discharge channel 3. The (bound) biological particles 11 or 13 are hence separated from the other biological materials 12.

FIG. 2 shows an immunomagnetic separation device, the basic construction of which corresponds to the separation device shown in FIG. 1. In the flow direction after the first electromagnet 6 and in front of the second electromagnet 7, the throughflow channel 5 has however a bulge (reaction chamber) 14 which is disposed on the side of the first electromagnet 6. In the present case, the throughflow channel 5 is configured in one piece with the reaction chamber 14. However the reaction chamber 14 can also be produced as a separate component at a corresponding opening in the throughflow channel 5. In the illustrated sectional plane (arrangement plane of the inlet channels 1, 2, of the outlet channels 3, 4 and of the two electromagnets 6, 7), the reaction chamber 14 has a Ω-shaped cross-section. At the top of the reaction chamber 14, a T-shaped flow breaker 15 is disposed in the throughflow channel 5 in the illustrated section. The flow breaker 15 is disposed at the top of the chamber 14 in the flow direction such that it engages merely in the first liquid flow of the mixed liquid 9 and diverts this liquid flow into the reaction chamber 14. By means of the reaction device which comprises the flow breaker 15 and the reaction chamber 14, the path of the first liquid flow 9 through the flow channel 5 is lengthened. Due to this reaction device, the length of stay of the first liquid flow 9 in the throughflow channel 5 is increased proportionally to the volume of the reaction chamber 14. As a result, an increased contact efficiency or extension of the time which is available for the immunomagnetic particles 8 to bind to the specific biological particles 11, is provided. The probability that an immune reaction takes place or that the immunomagnetic particles 8 bind is hence increased. The separation efficiency is hence increased by the increased immune-reaction efficiency of the device. The presented reaction chamber 14 causes high flow rate gradients and good micro-intermixing of the first liquid flow 9. As a result, also the binding probability of the immunomagnetic particles 8 is increased. It is hereby crucial that the reaction device 14, 15 is made available in the flow direction between the two electromagnets 6 and 7 so that the first liquid flow, if it already has the drawn-in immunomagnetic particles 8, is introduced into this reaction chamber 14 which extends the binding time period.

FIG. 3 shows a further separation device according to the invention which is configured extensively like that in FIG. 1. In contrast to FIG. 1 however, there is now situated between the inflow region E and the discharge region A which is disposed downstream thereof a separating wall 17 which separates the two liquid flows, which are supplied through the inlet channel 1 or the inlet channel 2 to the separating device, from each other. Thus it is possible merely in the region E of the two inlets 1 and 2 that the magnetic particles, due to the magnetic force exerted by the magnet 6, change from the one liquid flow into the other and the same exchange is effected in region A in the reverse direction. Between these two regions E and A, no further intermixing of the liquid flows can be effected so that, in this region, merely an agglomeration between immunomagnetic particles and antigen-attached particles is effected.

FIG. 4 shows a further separation device according to the invention. The supply of immunomagnetic particles 11 is effected here via an inlet channel 2 and the supply of the sample via an inlet channel 1, which particles communicate with each other in a region designated with E so that the immunomagnetic particles 11 can pass over into the sample due to an applied magnetic field Fmag. The magnetic field Fmag which is produced is represented by an arrow. The sample with the immunomagnetic particles 11 is then guided in a spiral 18 over a long path so that the immunomagnetic particles 11 can couple there with antigens 8. The spiral 18 is then guided back and, in a region A, meets the liquid which has been deflected in the meantime and contained the immunomagnetic particles 11 originally. In this region A, the particles 11 loaded with the immunoparticles 8 are in turn drawn back again into the original liquid flow by the magnetic field Fmag and subsequently are discharged via the outlet 4. The sample which is hence extensively freed again of the immunomagnetic particles 11 is guided in a large arc 19 around the spiral 18 and finally discharged via the outlet 3. This arrangement has the advantage that the mixing region between the magnetic particles 11 and the antibodies 8 has a very long path. Furthermore it has the advantage that merely one magnet is required in order to produce the magnetic field in the region E and the magnetic field in the region A and hence to effect all the mixing and separating processes.

1. Separation A separation device having a throughflow channel with a wall, an inflow region and a discharge region which is disposed, downstream thereof and at least one magnet for producing a magnetic field across at least a part of the cross-section of the throughflow channel, two inlet channels for the supply of fluids opening into the throughflow channel in the inflow region and two discharge channels for transporting fluids away leading out of the discharge region, the at least one magnet producing a magnetic field at a first location downstream of the inflow region, the at least one magnet also producing a magnetic field at a second location downstream of the first location and upstream of the discharge region, the magnetic field at the first and the second locations being essentially oppositely situated with respect to the direction of the stream or with respect to their polarity.

2. The separation device according to claim 1 wherein the at least one magnet comprises a first magnet for producing a first magnetic field at the first location and a second magnet for producing a second magnetic field at the second location, the first magnet being disposed downstream of the inflow region and at least one of the following locations: laterally without the throughflow channel; at least partially integrated in the wall of the throughflow channel; and, within the wall in the throughflow channel, the second magnet being disposed downstream of the first magnet and upstream of the discharge region and at least one of the following locations: laterally without the throughflow channel; at least partially integrated into the wall of the throughflow channel; and, within the wall in the throughflow channel, and the first and the second magnets being disposed substantially on opposite sides of the throughflow channel.

3. The separation device according to claim 2 wherein the two inlet channels, the two discharge channels and the first and second magnets are disposed essentially in one plane in the flow direction of the throughflow channel.

4. The separation device according to claim 1 wherein at least one of at least one of the inlet channels opens into and at least one of the outlet channels, leads away from the throughflow channel at an inclination angle α thereto, 0≦α<45°.

5. The separation device according to claim 1 wherein one of the inlet channels and one of the outlet channels are disposed on the same side of the throughflow channel when viewed perpendicularly to the flow direction of the throughflow channel.

6. The separation device according to claim 1 wherein the at least one magnet is at least one of a permanent magnet and an electromagnet.

7. The separation device according to claim 6 wherein the at least one magnet comprises an electromagnet and the at least one of the field strength and the field gradient of the electromagnet can be at least one of maintained constant and varied at least one of temporally and locally.

8. The separation device according to claim 1 wherein at least one of the throughflow channel and the inlet channels and the outlet channels are at least one of disposed such that and spatially configured such that, in the throughflow channel during throughflow of fluids which can be used for immunomagnetic separation, at least one of a laminar flow and a flow with a Reynolds' number R which is smaller than the critical Reynolds' number R_{crit} can be produced.

9. The separation device according to claim 8 wherein at least one of the throughflow channel and the inlet channels and the outlet channels are at least one of disposed and configured such that a first liquid flow can be formed in the throughflow channel on the side of the first magnet and that a second liquid flow which is separated from the first liquid flow apart from diffusion processes can be formed in the throughflow channel on the side of the second magnet.

10. The separation device according to claim 1 wherein the throughflow channel is a microfluid channel with a cross-sectional area perpendicular to the throughflow direction between about 0.002 mm² and about 1 mm².

11. The separation device according to claim 1 wherein the throughflow channel is a tube which in cross-section is one of substantially circular, substantially elliptical, substantially rectangular and substantially square.

12. The separation device according to claim 2 further including a reaction device disposed in the flow direction after the first magnet and before the second magnet, the reaction device lengthening the flow path.

13. The separation device according to claim 12 wherein the flow path-lengthening reaction device has a reaction chamber and a flow breaker which is disposed in the interior of the throughflow channel.

14. The separation device according to claim 13 wherein a first liquid flow can be directed into the reaction chamber with the flow breaker.

15. The separation device according to claim 14 wherein at least one of: in a plane parallel to the flow direction of the throughflow channel the reaction chamber has a cross section which is one of substantially Ω-shaped, substantially semicircular and substantially trapezoidal; and, in a plane parallel to the flow direction of the throughflow channel the flow breaker has a cross section which is one of substantially triangular and substantially T-shaped.

16. The separation device according to claim 13 wherein the reaction chamber comprises one of a bulge in the wall of the throughflow channel, one piece of the throughflow channel, and a separate component which is disposed at an opening of the wall of the throughflow channel.

17. The separation device according to claim 2 further comprising at least one of a control device for controlling at least one of the first and second magnets and a regulating device for regulating at least one of the throughflow rate in the throughflow channel, the throughflow rate in the inlet channels and the throughflow rate in the discharge channels.

18. The separation device according to claim 1 adapted for at least one of: implantation in at least one of a human body and an animal body; and, use outside of at least one of a human body and an animal body.

19. The separation device according to claim 1 wherein a separating wall is disposed at least in regions in the throughflow channel between the region of the inlet channels and the region of the discharge channels in the flow direction of the fluid, said separating wall preventing intermixing of adjacent fluid flows introduced separately from each other.

20. A separation arrangement comprising a separation device according to claim 1 and a fluid which has a plurality of at least one of immunomagnetic particles, antibody-coupled particles and particles which are coupled with antigen-specific tetramers.

21. The separation arrangement according to claim 20 wherein the particles have at least one of ferromagnetic properties, superparamagnetic properties and a substantially spherical shape.

22. The separation arrangement according to claim 20 wherein at least one of: at least one of the throughflow channel, the inlet channels and the outlet channels of the separation device are configured such that; and, the fluid has a viscosity, density, temperature and average flow rate in the throughflow channel such that, in the throughflow channel, at least one of a laminar flow and a flow with a Reynolds' number R which is smaller than the critical Reynolds' number R_{crit} is present.

23. The separation arrangement according to claim 20 wherein the throughflow rate of the fluid which has the particles through the throughflow channel is between about 0.1 μl/min and about 2000 μl/min.

24. The separation arrangement according to claim 20 wherein the average throughflow rate of the fluid which has the particles in the throughflow channel is between about 0.03 mm/s and about 3000 mm/s.

25. A method for the isolation of a specific biological material from a first fluid including the biological material, the method comprising providing a second fluid which has a plurality of immunomagnetic particles, introducing the first fluid and the second fluid into a throughflow channel such that, in the throughflow channel, laminar flow conditions are formed between the first fluid flow and the second fluid flow through the throughflow channel, applying a magnetic field to the second fluid flow, drawing the immunomagnetic particles at least partially from the second fluid flow into the first fluid flow with the help of the magnetic field, leaving the immunomagnetic particles drawn into the first fluid flow in the first fluid flow in order to bind to the biological material over a binding period of time, applying a magnetic field to draw the immunomagnetic particles bound to the biological material at least partially from the first fluid flow into the second fluid flow, and separately discharging the two liquid flows from the throughflow channel.

26. The method according to claim 25 performed with the device of claim 20.

27. The method according to claim 25 wherein applying a magnetic field includes applying a first magnetic field having at least one of a field strength and a gradient strength that is just sufficient for the transfer of the immunomagnetic particles from the second into the first fluid flow, and applying a magnetic field further includes applying a second magnetic field having at least one of a field strength and a gradient strength that is just sufficient for the transfer of the immunomagnetic particles which are bound at least partially to the biological material from the first fluid flow into the second fluid flow.

28. The method according to claim 25 wherein at least one of applying a magnetic field to the second fluid flow and applying a magnetic field to draw the immunomagnetic particles bound to the biological material at least partially from the first fluid flow comprises at least one of applying a pulsed magnetic field and applying a sinusoidally modulated magnetic field.

29. The method according to claim 25 wherein leaving the immunomagnetic particles drawn into the first fluid flow in the first fluid flow in order to bind to the biological material over a binding period of time comprises extending the first fluid flow to increase the binding period of time.

30. The method according to claim 25 performed at least one of outside and within at least one of a human body and an animal body.

31. The method of claim 25 for performing at least one of medical diagnosis and therapy at least one of outside and within at least one of a human body and an animal body.

32. The method of claim 25 wherein the specific biological material comprises an antigen and the plurality of immunomagnetic particles are coupled with at least one of antibodies, tetramers and streptamers which are specific to the antigen.
